(19) United States
(12) Patent Application Publication
(10) Pub. No.: US 2025/0288357 A1
(43) Pub. Date: Sep. 18, 2025

(54) MODELING AND MEASUREMENT OF STENT DEVICES

(71) Applicant: Covidien LP, Mansfield, MA (US)

(72) Inventors: Adam Hendry, San Clemente, CA (US); Ajay Kadrenahally, Rancho Santa Margarita, CA (US)

(21) Appl. No.: 19/078,022

(22) Filed: Mar. 12, 2025

Related U.S. Application Data
(60) Provisional application No. 63/564,427, filed on Mar. 12, 2024.

(51) Int. Cl. A61B 34/10; A61B 17/12

(52) U.S. Cl. CPC A61B 34/10; A61B 17/12113; A61B 17/12172; A61B 2034/102; A61B 2034/108

(57) ABSTRACT

A method for modeling a device includes receiving a surface envelope of the device, receiving helicoids corresponding to a pitch of the device, generating wire centerline paths based on the surface envelope and the helicoids, generating wire intersection points, generating wire intersection normals that are normal to the surface envelope at the wire intersection points, shaping the wire centerline paths by warping the wire intersection points along the wire intersection normals in accordance with a braid pattern, and generating a braid model of the stent device based on the shaped wire centerline paths and a wire diameter. Furthermore, a method for characterizing a device includes receiving a surface envelope of the device, generating surface envelope normals that are normal to the surface envelope, generating a masked surface envelope characterizing a wire pattern of the device, and determining dimensional attributes of the device based on the masked surface envelope.

### CROSS-REFERENCE TO RELATED APPLICATIONS

This application claims the benefit of and priority to U.S. Provisional Patent Application Ser. No. 63/564,427 filed Mar. 12, 2024, the entire disclosure of which is incorporated by reference herein.

### TECHNICAL FIELD

The present technology relates to the modeling and measurement of stent devices, including the modeling and measurement of stent devices having developable or non-developable surfaces.

### BACKGROUND

Stents are medical devices that are commonly positioned in patients to treat various vascular conditions. For example, an intravascular stent can be delivered to an occlusion (e.g., thrombus) in a vessel in a mechanical thrombectomy procedure to engage and remove the occlusion, or implanted in a lumen of a vessel to help prop open the lumen of the vessel. As another example, an intrasaccular stent can be positioned within an aneurysm to close the aneurysm neck from inside the aneurysm, thereby treating the aneurysm through intrasaccular flow disruption. Many stents are mesh or braided structures with properties that can vary according to changes in the vascular environment in which they are placed or otherwise used, such as across different patients.

Understanding how stent dimensional and mechanical attributes change according to changes in vascular geometry is important for procedural planning and patient outcomes. There exist various computational techniques, such as finite element analysis (FEA), fluid-structure interaction (FSI) analysis, and computational fluid dynamics (CFD) analysis, to study and predict the behavior of stents that cannot be quantified in-vivo. Such computational techniques also can be used to optimize stents by iteratively testing and changing design parameters. Accurate input models of the stent of interest are an important first step to obtaining reliable analytical results. However, traditional computer-aided drawing (CAD) packages are incapable of generating braided structures to the level of accuracy and detail required for computational analysis.

Furthermore, accurate knowledge of stent dimensional attributes such as metal surface area and porosity is also important, as regulatory bodies (e.g., Food and Drug Administration) may require such information for regulatory product submissions, and physicians may rely on such stent dimensional attributes for insight into product performance. Some stent measurement systems (e.g., digital microscopes) exist, but such devices cannot reliably measure desired stent properties. For example, many stents have high degrees of 3D curvature (e.g., have non-developable geometries) and/or other geometrical complexities such as being dual-layered or having morphologies that change continuously along the stent surface, and existing measurement systems are insufficient.

Accordingly, there is a need for new and improved techniques for the modeling and measurement of stent devices.

### SUMMARY

The subject technology is illustrated, for example, according to various aspects described below, including with reference to FIGS. 1-16. Various examples of aspects of the subject technology are described as numbered clauses (1, 2, 3, etc.) for convenience. These are provided as examples and do not limit the subject technology.

Example 1. A method for modeling a braided stent device, the method comprising: receiving a surface envelope mesh of the stent device; receiving at least one helicoid corresponding to a braid pitch of the stent device; generating a plurality of wire centerline paths based on the surface envelope mesh and the helicoids; generating a plurality of wire intersection points corresponding to intersections of the wire centerline paths; generating a plurality of wire intersection normal vectors that are normal to the surface envelope mesh at the wire intersection points; shaping the wire centerline paths by warping the wire intersection points along the wire intersection normal vectors in accordance with a desired braid pattern; and generating a braid model of the stent device based on the shaped wire centerline paths and a desired wire diameter.

Example 2. The method of clause 1, wherein the at least one helicoid surface comprises a right-hand helicoid and a left-hand helicoid.

Example 3. The method of clause 1 or 2, wherein generating the plurality of wire centerline paths comprises determining intersections between the surface envelope mesh and the helicoid surfaces.

Example 4. The method of any one of clauses 1-3, further comprising modifying the wire centerline paths to include ordered, equispaced wire centerline points.

Example 5. The method of clause 4, wherein generating the plurality of wire intersection normal vectors comprises: generating a plurality of surface envelope normal vectors that are normal to the surface envelope mesh; and defining the plurality of wire intersection normal vectors as a subset of surface envelope normal vectors located at the wire intersection points.

Example 6. The method of clause 5, wherein generating a plurality of surface envelope normal vectors comprises: determining, for each point on the surface envelope mesh, a best fit plane through the point that minimizes the distance between the point and the k-nearest neighbors of the point; determining, for each point on the surface envelope mesh, a surface envelope normal vector that is normal to the best fit plane corresponding to the point; and orienting the surface envelope normal vectors such that all of the surface envelope normal vectors point inward or outward relative to the surface envelope mesh.

Example 7. The method of any one of clauses 1-6, wherein generating a braid model of the stent device comprises sweeping wire surfaces with the desired wire diameter around the shaped wire centerline paths.

Example 8. A method for characterizing a braided stent device, comprising: receiving a surface envelope mesh of the stent device, the surface envelope mesh comprising representations of wires of the stent device; generating a plurality of surface envelope normal vectors that are normal to the surface envelope mesh; generating a masked surface envelope mesh characterizing a wire pattern of the device, based on the surface envelope mesh and the plurality of surface envelope normal vectors; and determining one or more dimensional attributes of the stent device based on the masked surface envelope mesh.

Example 9. The method of clause 8, wherein generating a plurality of surface envelope normal vectors comprises: determining, for each point on the surface envelope mesh, a best fit plane through the point that minimizes the distance between the point and the k-nearest neighbors of the point; determining, for each point on the surface envelope mesh, a surface envelope normal vector that is normal to the best fit plane corresponding to the point; and orienting the surface envelope normal vectors such that all of the surface envelope normal vectors point inward or outward relative to the surface envelope mesh.

Example 10. The method of clause 8 or 9, wherein generating a masked surface envelope mesh comprises: projecting the surface envelope normal vectors via ray tracing; generating a mask of cells comprising a plurality of wire cells and a plurality of open cells, wherein the plurality of wire cells are located where the projected surface envelope normal vectors intersect with the representation of wires of the stent device, and wherein the plurality of open cells are located where the projected surface envelope normal vectors do not intersect with the representation of wires of the stent device.

Example 11. The method of any one of clauses of 8-10, wherein determining one or more dimensional attributes of the stent device comprises determining at least one of: porosity, coverage factor, pore size, pore density, braid angle, pitch, or amount of foreshortening of the stent device.

Example 12. The method of any one of clauses 8-11, wherein determining one or more dimensional attributes of the stent device comprises determining porosity by: generating a plurality of two-dimensional contours from the masked surface envelope mesh that are normal to a longitudinal axis of the device; for each contour, determining a percent open arclength of the contour, the percent open arclength corresponding to one or more open cells of the masked surface envelope mesh in the contour; and determining porosity of the stent device based on the sum of the percent open arclengths among the contours.

Example 13. The method of any one of clauses 8-12, wherein determining one or more dimensional attributes of the stent device comprises determining coverage factor by: generating a plurality of two-dimensional contours from the masked surface envelope mesh that are normal to a longitudinal axis of the device; for each contour, determining a percent wire arclength of the contour, the percent wire arclength corresponding to one or more wire cells of the masked surface envelope mesh in the contour; and determining porosity of the stent device based on the sum of the percent wire arclengths among the contours.

Example 14. The method of any one of clauses 8-13, wherein determining one or more dimensional attributes of the stent device comprises determining pore size by: isolating the open cells in the masked surface envelope mesh; segmenting the isolated open cells into individual pores of the stent device using connectivity analysis; and determining pore size based on the sum of surface areas of the individual pores in the segmented cells.

Example 15. The method of any one of clauses 8-14, wherein determining one or more dimensional attributes of the stent device comprises determining pore density of a region of interest by: isolating the wire cells in the masked surface envelope mesh for at least the region of interest; determining wire centerlines of at least the region of interest by skeletonizing the isolated wire cells; slicing the masked surface envelope mesh along the wire centerlines; segmenting the isolated wire cells into individual pores of the stent device using connectivity analysis; and determining pore density of the region of interest based on the number of pores in the region of interest divided by the surface area in the region of interest.

Example 16. The method of any one of clauses 8-15, wherein determining one or more dimensional attributes of the stent device comprises determining braid angle of the stent device by: isolating the open cells in the masked surface envelope mesh; segmenting the isolated open cells into individual pores of the stent device using connectivity analysis; and determining outer boundary edges of the segmented open cells and one or more intersection points of the outer boundary edges; determining the braid angle based on a dot product between outer boundary edges located at each of the one or more intersection points.

Example 17. The method of any one of clauses 8-16, wherein determining one or more dimensional attributes of the stent device comprises determining pitch of the stent device by: isolating the open cells in the masked surface envelope mesh; segmenting the isolated open cells into individual pores of the stent device using connectivity analysis; determining outer boundary edges of the segmented open cells and one or more intersection points of the outer boundary edges; and determining pitch as a distance between adjacent intersection points along a longitudinal axis of the stent device.

Example 18. The method of any one of clauses 8-17, further comprising generating the surface envelope mesh from imaging data.

Example 19. The method of any one of clauses 8-18, comprising providing an output comprising the one or more dimensional attributes of the stent device.

Example 20. The method of clause 19, wherein the output comprises at least one of a written report, a graphical display of the stent device, a computer-aided drawing model, or a data format for finite element analysis.

Example 21. The method of any one of clauses 1-20, wherein the stent device is an intravascular stent device.

Example 22. The method of any one of clauses 1-20, wherein the stent device is an intrasaccular stent device.

Example 23. The method of any one of clauses 1-20, wherein the stent device has a non-developable surface.

Example 24. A system comprising: a processor; and a memory operably coupled to the processor and storing instructions that, when executed by the processor, cause the computing system to perform operations comprising the method of any one of clauses 1-23.

Example 25. A non-transitory computer-readable storage medium comprising instructions that, when executed by one or more processors of a computing system, cause the computing system to perform operations comprising the method of any one of clauses 1-23.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates an example braid pattern of an intravascular stent device with a developable surface.

FIG. 2 illustrates an example intrasaccular stent device with a non-developable surface.

FIG. 3 is a schematic illustration of a device modeling and measurement system.

FIGS. 4A and 4B are flowchart schematics of an example method for modeling a braided stent device, in accordance with the present technology.

FIG. 5 is a flowchart schematic of an example method for measuring one or more dimensional attributes of a stent device, in accordance with the present technology.

FIG. 6 is a flowchart schematic of an example method for generating a surface envelope mesh of a stent device, in accordance with the present technology.

FIG. 7 is a schematic illustration of a braided stent device and various dimensional attributes of the stent device.

FIG. 8-13 are flowchart schematics of example methods for determining various types of dimensional attributes of a stent device based on a masked surface envelope mesh, in accordance with the present technology.

FIGS. 14A-14C illustrate portions of an example implementation of the method for modeling a braided stent device, in accordance with the present technology.

FIG. 15 illustrates various example displayed outputs relating to measured pore size of a stent device, in accordance with the present technology.

FIG. 16 illustrates various example displayed outputs relating measured porosity of a stent device, in accordance with the present technology.

### DETAILED DESCRIPTION

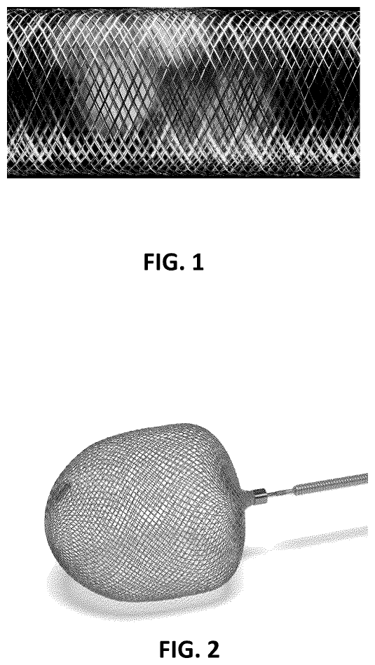

The present technology relates to modeling and measurement of medical devices, such as stent devices. Some variations of the present technology, for example, are directed to the modeling and measurement of braided stents. Specific details of several variations of the technology are described below with reference to FIGS. 1-16.

### I. STENT GEOMETRIES

Described herein are various modeling and measurement techniques to model and otherwise characterize stent devices that can be positioned (e.g., operated and/or implanted) in a body for medical treatment. For example, such modeling and measurement techniques can be used to characterize braided stents that are formed from braided wires (e.g., metal wires). Braided stents can have a variety of geometries, which can depend, for example, on the type of application for their use, the type of anatomical environment in which they are placed, and/or anatomical variation among patients as stents can be sized and/or shaped appropriately. In some variations, a braided stent can be tubular, as shown for example in FIG. 1. For example, tubular stents can be particularly well-suited as intravascular stents (e.g., for placement in a blood vessel for mechanical thrombectomy or treatment of an aneurysm while placed outside the aneurysm). In many instances, some or all of a tubular stent can be characterized as having a developable surface geometry (i.e., a smooth surface with zero Gaussian curvature, that can be flattened onto a plane without distortion). While tubular devices may be characterized to some extent based on closed-form theoretical equations, the accuracy of such analysis may be limited to mechanically braided technology, and cannot be used to model hand-woven braids that inherently exhibit minor variations stent-to-stent. Furthermore, traditional measurement techniques relying on theoretical equations are not suitable for even tubular devices that are implanted in curved anatomy However, the modeling and measurement techniques described herein can be used to characterize tubular devices and other devices with developable surfaces with greater accuracy, as well as a greater range of tubular devices including mechanically braided and hand-woven braided stents.

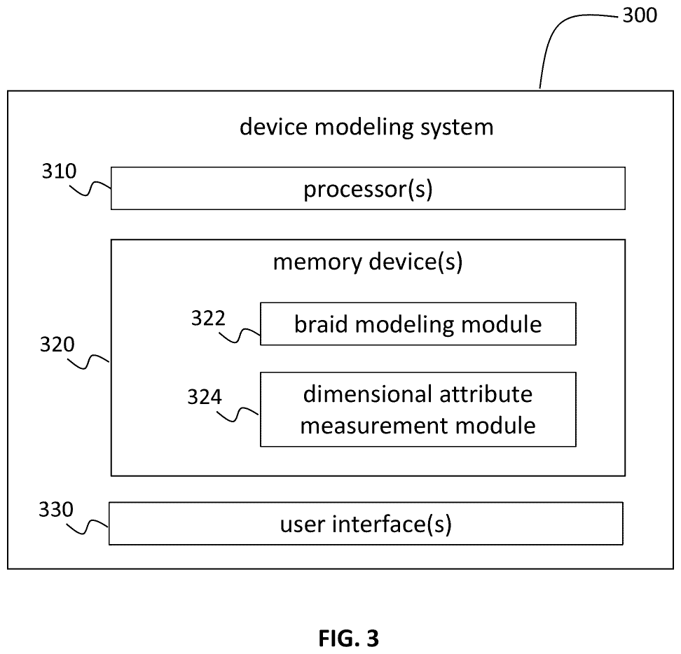

In some variations, a braided stent can have more complex geometries including high degrees of 3D curvature and/or dual layers. For example, a braided stent can be characterized as having a non-developable surface (also known as a surface with “double curvature”, “compound curvature”, or “non-zero Gaussian curvature”). Many intrasaccular stents (e.g., for placement within an aneurysm) have non-developable surfaces. For example, FIG. 2A illustrates the Artisse™ intrasaccular device (available from Medtronic), which is an expanding, braided ovoid implant made of a double layer of nitinol wire mesh. Other examples of intrasaccular devices with non-developable surfaces include the Contour Neurovascular System™ available from Stryker™ and the WEB™ Embolization System available from MicroVention™. Such non-developable surfaces are greatly more complex than developable surfaces to model and analyze due to their complexities. However, the modeling and measurement techniques described herein can be used to characterize non-developable surfaces that other existing techniques cannot.

### II. DEVICE MODELING AND MEASUREMENT

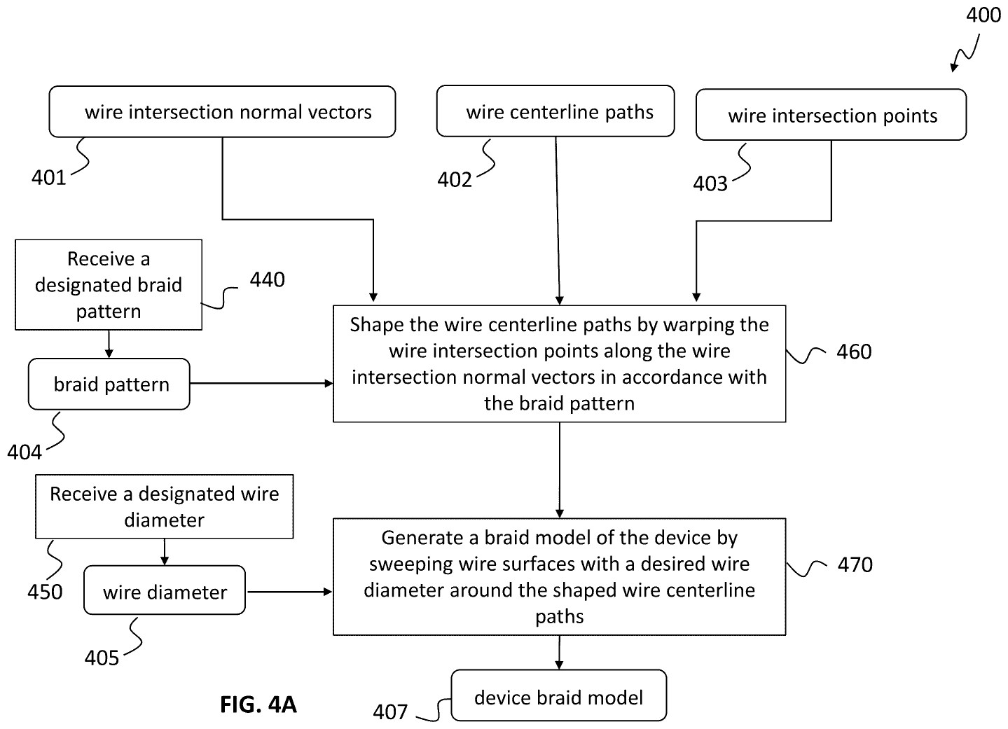

FIG. 3 is a schematic diagram of an example device modeling and measurement system 300. As shown in FIG. 3, the device modeling and measurements system can include one or more processors 310, and one or more memory devices 320 having instructions stored therein. The one or more memory devices 320 can include any suitable computer-readable medium such as RAMs, ROMs, flash memory, EEPROMs, optical devices (e.g., CD or DVD), hard drives, floppy drives, or any suitable device. The memory device 320 may include instructions corresponding to a braid modeling module 322 and/or a dimensional attribute measurement module 324. The memory device 320 may include instructions for performing modeling and measurement methods such as those described herein.

The processor 310 may be configured to execute the instructions that are stored in the memory device 320 such that, when it executes the instructions, the processor 310 perform aspects of the methods herein. The instructions may be executed by computer-executable components integrated with a software application, applet, host, server, network, website, communication service, communication interface, hardware, firmware, software elements of a user computer or mobile device, smartphone, or any suitable combination thereof. In some variations, the one or more processors 310 can be incorporated into a computing device or system such as a cloud-based computer system, a mainframe computer system, a grid-computer system, or other suitable computer system.

The device modeling and measurement system 300 can further include one or more user interfaces 330, which functions to allow a user to interact with the device modeling system 300. For example, the user interface 330 can be configured to receive user input (e.g., for controlling input of information into the braid modeling module 322 and/or dimensional attribute measurement module 324 for characterizing a stent device) and/or provide information to a user. For example, in some embodiments the user interface 330 can include a display (e.g., monitor) for displaying a model and/or one or more dimensional attribute measurements of a stent device.

The device modeling and measurement system 300 can be used to generate a braid model and/or measure one or more dimensional attributes of a stent device. The system 300 can advantageously function to model and/or measure a wide range of stent devices, regardless of device manufacturer. Furthermore, such characterization of stent devices can be largely automated, with minimal user input, thereby providing for modeling and measurements systems and methods that are highly intuitive and easy to use by engineers, medical professionals, and other users. In some variations, the modeling and/or measurement techniques in accordance with the present technology can be used to help improve the design process (e.g., iterative design), and/or regulatory submissions (e.g., FDA submissions) by standardizing and/or improving accuracy of stent characterization.

In some variations, the stent device can be a braided stent device formed from metal wires. For example, the braided stent device can include woven or otherwise braided wires including a shape memory alloy (e.g., nitinol). However, the stent device can additionally or alternatively include any other metal such as a stainless steel, etc. Furthermore, the stent device can additionally or alternatively include wires of any suitable material (e.g., polymer filaments). The braided stent device can be machine-braided and/or hand-braided.

In some variations, the stent device can be configured to self-expand from a radially compressed configuration (e.g., with a first diameter, such as for delivery purposes) to a radially expanded configuration (e.g., with a second diameter larger than the first diameter, such as for placement in a patient), and/or the stent device can be actively expanded from the radially compressed configuration to the radially expanded configuration with an expandable device (e.g., balloon). The stent device that is modeled and/or measured using the system 300 can include a developable surface (e.g., tubular) and/or include a non-developable surface (e.g., ovoid, toroidal, dual-layered, etc.).

In some variations, the stent device is an intravascular stent (e.g., for mechanical thrombectomy), while in some variations the stent device is an intrasaccular stent (e.g., for treatment of an aneurysm). The stent device can be configured for placement in any suitable anatomical region, such as a neurovascular region (e.g., cerebral artery), a cardiac region (e.g., coronary artery), and/or any suitable treatment location.

### A. Braid Modeling

Example aspects of methods of modeling a braided stent device are described in further detail below, and can be incorporated in the braid modeling module 322.

Generally, a method for modeling a braided stent device can include receiving a set of basic input information about the stent device, including a surface envelope mesh corresponding to the wire centerlines of the stent device and a pair of helicoids (e.g., right-hand and left-hand helicoids) corresponding to the braid pitch of the stent device. From this basic information, further information including wire centerline paths (path of the braid wires taken along the centerlines of the braid wires), wire intersection points (located where braid wires intersect), and wire intersection normal vectors (normal surface vectors located at the wire intersection points) can be generated and subsequently used in creating the braid model of the stent device. Rather than generating braid models using closed-form equations, the method generally uses a surface envelope mesh of the stent device to automatically calculate wire weave directions, etc. of the stent device.

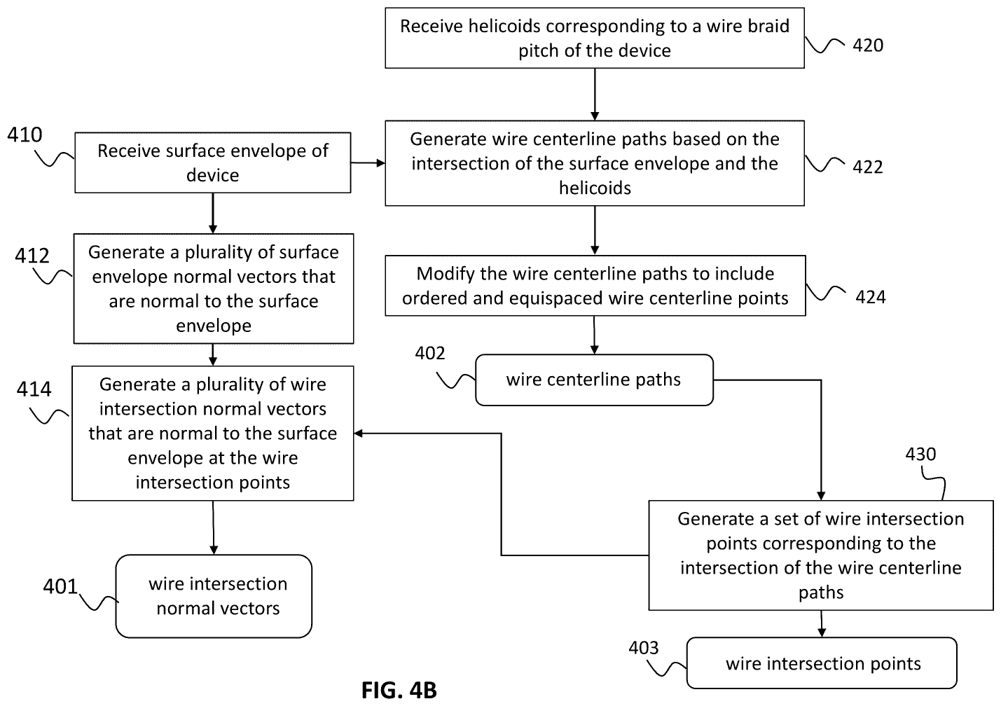

For example, with reference to FIG. 4A, a method 400 for modeling a braided stent device can include generating a plurality of wire centerline paths 402 for the stent device, generating a plurality of wire intersection points 403 corresponding to intersections of the wire centerline paths, and generating a plurality of wire intersection normal vectors 401 that are normal to a surface envelope mesh at the wire intersection points. The method 400 can further include receiving a desired or designated braid pattern for the stent device 440, and receiving a desired or designated wire diameter for the stent device 450. The wire centerline paths 402, wire intersection points 403, wire intersection normal vectors 401, braid pattern 404, and wire diameter 405 can be used to generate a braid model of the stent device. For example, the method 400 can further include shaping the wire centerline paths by warping the wire intersection points along the wire intersection normal vectors in accordance with the braid pattern 460, and generating a braid model of the stent device by sweeping wire surfaces with the wire diameter around the shaped wire centerline paths 470. The output of the method 400 can be a device braid model 407, which can, for example, be viewed and/or manipulated by a user (e.g., in computer-aided drawing software), and/or provided to suitable analytical software (e.g., finite element analysis software) for further characterization of the stent device. Further details of the method 400, including example variations of how the wire centerline paths 402, wire intersection points 403, and wire intersection normal vectors 401 can be generated, are described below with reference to FIGS. 4A and 4B.

### 1. Surface Envelope Mesh and Helicoids

As shown in FIG. 4B, the method 400 for modeling a braided stent device can include receiving a surface envelope of the stent device 410. In some variations, for purposes of generating a braid model, the surface envelope corresponds to the wire centerlines; that is, the envelope represents a surface profile of the stent device taken along the center of the wires forming the braided stent device. The surface envelope can function as an initial input for the method 400. In some embodiments, the surface envelope can be a file export of a surface envelope mesh (e.g., with triangular cells) from a suitable computer-aided drawing (CAD) software application.

The method 400 can further include receiving helicoids corresponding to a wire braid pitch of the stent device 420. For example, the received helicoids can include a right-hand helicoid surface and a left-hand helicoid surface that are representative of the braid pitch of the stent device. The helicoids can function as another initial input for the method 400. In some embodiments, the helicoids can be a file export from a suitable CAD software application, or can be automatically derived from a CAD model of the stent device.

### 2. Generating Wire Centerline Paths

As shown in FIG. 4B, the method 400 can further include generating wire centerline paths 422. The wire centerline paths can be generated by determining where the surface envelope and one or more of the helicoids intersect. The surface envelope and helicoids intersect at a plurality of point locations, and such point locations collectively form intersection curves representing the wire centerline paths. Mathematically, the surface envelope and the helicoids are implicit functions. For example, the surface envelope may be conceptually represented by an implicit function F=0, while the helicoids may be conceptually represented by another implicit function G=0. Accordingly, determining the intersection between the surface envelope and the helicoids can be performed by numerically solving a system of equations where F−G=0. The wire centerline paths 402 may be identified as the intersection curves determined through this analysis.

Once the wire centerline paths are generated (e.g., through determination of the intersection curve described above), points in the intersection curves may not be equally spaced apart, and/or may not be properly oriented (ordered) from the beginning to the end of each curve. Accordingly, the method 400 can further include modifying the wire centerline paths to include ordered, equispaced wire centerline points 424. The modified wire centerline paths 402 with ordered, equispaced wire centerline points can then be used in the development of a braid model as described herein.

In some variations, topology can be implemented to orient an intersection curve to have ordered wire centerline points. Each intersection curve can be considered as an open graph with two nodes of valence 1 (i.e., the two end points of the intersection curve). Connectivity information regarding how the various nodes are connected to each other may be determined during the process of generating the wire centerline paths 422 (e.g., when generating the intersection curves themselves). In other words, once the intersection curves are generated, it is known which other nodes each node is connected to. Accordingly, intersection curves can be “traversed” based on such connectivity information to identify the order in which points along the wire centerline points are connected. For example, to order points on the wire centerline paths from one end node of the intersection curve to the other end of the intersection curve, the nodes with valence 1 (i.e., corresponding to end nodes of the intersection curve) can first be identified. Starting with one node as a start node (e.g., based on user input, or default selection by the system), an intersection curve can be traversed from the start node to the other connected node of valence 1 (i.e., end node) using the known connectivity information from the intersection curve. Through this traversal, the order in which the wire centerline points are connected is identified, and used to modify or generate a properly oriented set of wire centerline points for each intersection curve.

In some variations, a suitable statistical resampling algorithm can be used to equispace the wire centerline points. For example, an intersection curve can be considered to include a plurality of line segments, each of which has an arclength and an orientation given by the vector of the line segment pointing from one wire centerline point to the next (e.g., among wire centerline points oriented in a manner such as that described above). An example statistical resampling process includes receiving a desired number of wire centerline points (and/or desired spacing between wire centerline points, from which the desired number of wire centerline points can be calculated for a given curve length) on an intersection curve, which can, for example, be a user input or a default setting in the braid modeling module. The initial intersection curve prior to resampling is arclength parametrized and normalized (e.g., normalized to 1, with 0 representing a start point of the intersection curve, and 1 representing an end point of the intersection curve). The normalized intersection curve is then arclength parametrized with the desired number of new wire centerline points equispaced across the normalization range (e.g., between 0 and 1, if normalized to 1). In some instances, any new wire centerline point may lie between two older, initial wire centerline points, so a histogram of where the new centerline points are located relative to the initial intersection curve can be created. Accordingly, the bins representing where the new wire centerline points are located in the initial intersection curve can be calculated. The location of each new wire centerline point within a particular bin can be determined in accordance with Equation 1 below:

xₙ=start_point+t*s·T (1)

where t is the vector from the start point of the intersection curve to the end point of the intersection curve within the bin, and s is the arclength from the start point to the new wire centerline point in that bin.

### 3. Generating Wire Intersection Points

As shown in FIG. 4B, the method 400 can further include generating a set of wire intersection points 430. The wire intersection points 403 generated through this process are wire centerline points that correspond to (are located at) the intersection of the various wire centerline paths. Once the wire centerline points are equispaced and oriented in a manner such as that described above, such a set of wire intersection points 403 can be generated and used to develop a set of wire intersection normal vectors 401 as described in further detail below.

Mathematically, the intersection curves for the wire centerline paths are implicit functions. For example, a first intersection curve may be conceptually represented by an implicit function M=0, while a second intersection curve may be conceptually represented by an implicit function N=0. Accordingly, determining the intersection between the first and second intersection curves can be performed by numerically solving a system of equations where M-N=0. The wire intersection points 403 may be identified as the points of intersection determined through this analysis.

### 4. Generating Wire Intersection Normal Vectors

As shown in FIG. 4B, the method 400 can further include generating a plurality of surface envelope normal vectors that are normal to the surface envelope of the device 410, and generating a plurality of wire intersection normal vectors 414 (which can, for example, be a subset of the surface envelope normal vectors). The wire intersection normal vectors 401 generated through this process are vectors that are normal to the surface envelope at the wire intersection points 403.

In some variations, generating a plurality of surface envelope normal vectors 412 can include implementing a version of Hoppe's algorithm (see Hoppe et al., “Surface Reconstruction from Unorganized Points”, ACM SIGGRAPH Computer Graphics, Volume 26, Issue 2, 71-78 (Jul. 1, 1992)). For example, generating the surface envelope normal vectors can include, for each point on the surface envelope mesh, finding the best fit plane through the point that minimizes the distance between it and its k-nearest neighbors, and computing the normal to this plane using principal component analysis (PCA). The normals on the surface are then oriented such that all the normals point outwards from the surface envelope or all the normal inwards from the surface envelope. This orientation can include (i) creating a Riemann graph through all points on the surface envelope where the edge weight is the Euclidean distance between the points connected by the edge, (ii) arbitrarily selecting one point's normal as having the “correct” orientation (e.g., the normal of a “control point” with the highest z-value, highest y-value, or highest x-value) and (iii) traversing the Riemann graph from the initial node/point of the Riemann graph to the final node/point of the Riemann graph, taking the dot product between the normal of the control point and the normal of each of various second nodes/points in the Riemann graph in a pairwise manner. In (iii), if the dot product is positive, this indicates that the normals are pointing in the same directions (both outward from the surface envelope or both inward from the surface envelope). However, if the dot product is negative, this indicates that the normal are pointing in the opposite directions, so the normal of the second node can be reversed (flipped 180 degrees) so that it matches the orientation of the control point's normal. When the traversal of the Riemann graph of (iii) is complete, the surface envelope normal vectors should all be oriented consistently outward or inward from the surface envelope. Either orientation is acceptable, as a user (or default setting) may furthermore re-adjust all of the surface envelope normals to be oriented outward or oriented inward, following the completion of (iii).

Given the plurality of surface envelope normal vectors and the wire intersection points 403 generated previously, a subset of normals that are located at the wire intersection points can be identified as wire intersection normals 401. For example, generating a plurality of wire intersection normals 414 can include performing a KDTree analysis to find the point on the surface envelope that is nearest each wire intersection point, and assign that point's surface envelope normal to the wire intersection point as that wire intersection point's corresponding normal. In other words, the set of wire intersection normals 401 can be those surface envelope normals that are nearest to the generated set of wire intersection points 403.

### 5. Shaping Wire Centerline Paths

As shown in FIG. 4A, given the determination of wire centerline paths 402, wire intersection points 403, and wire intersection normals 401, the method 400 can include shaping the wire centerline paths in accordance with a designated or desired braid pattern 460. For example, in some variations, the method 400 can include receiving a designated braid pattern 440 (e.g., as a user input, as a default setting, etc.). Shaping the wire centerline paths can include warping the wire intersection points 403 (e.g., over, under, etc.) along the wire intersection normals in accordance with the braid pattern. In some variations, it may be assumed that linearly spacing the points between wire intersection points is sufficient to result in an adequately accurate braid model. Examples of braid patterns 404 include one-over-one-under, two-over-two-under, other standard braid patterns, etc. Standard braid patterns can, for example, be identified by a unique code or other input. Furthermore, in some variations a braid pattern 404 can be described and/or referred to in a table or other suitable reference including a custom hand braiding pattern. However, the braid pattern can be designated or input in any suitable manner. Once shaped, the wire centerline paths can be undulating in accordance with the “over” and “under” nature of the braid pattern 404.

6. Generating Braid Model from Shaped Wire Centerline Paths

As shown in FIG. 4A, the method 400 can further include generating a braid model of the device based on the shaped, undulating wire centerline paths 470 and a designated or desired wire diameter. The method can, in some variations, include receiving a designated wire diameter 450 (e.g., as a user input, as default setting, etc.). For example, generating a braid model can include sweeping a surface with a designated or desired wire diameter 407 through the wire centerlines to generate the braid wires. Collision detection between wires may be used to ascertain whether wires should be warped more or less. Additionally or alternatively, given a potential assumption that the stent device is made from a fixed length of wire, the total length of the wire path in the braid model can be verified against a known (e.g., user-entered) fixed wire length.

### 7. Providing an Output of the Braid Model

In some variations, the method 400 can further include providing an output of the generated braid model of the stent device. For example, the output can be provided to a display (e.g., for viewing by a user) and/or communicated to a separate software application for further use (e.g., CAD software, FEA, manufacturing device such as 3D printing, etc.).

For example, the output can include a visual representation of the braid model. For example, the output can include an image of the braid model. The image can be a static image, or can include an animation (e.g., GIF). In some variations, the image can be manipulable by a user through the user interface (e.g., rotate, zoom in, zoom out, click and drag, etc.) to allow inspection of the braid model.

In some variations, the output can additionally or alternatively have a file format suitable for further analysis by CAD software, FEA software (e.g., ABAQUS input deck), ANSYS or other fluid dynamics package.

However, the output of the braid model can be provided in any suitable manner.

### B. Dimensional Attribute Measurement

Example aspects of methods of measuring one or more dimensional attributes of a braided stent device are described in further detail below, and can be incorporated in the dimensional attribute measurement module 324.

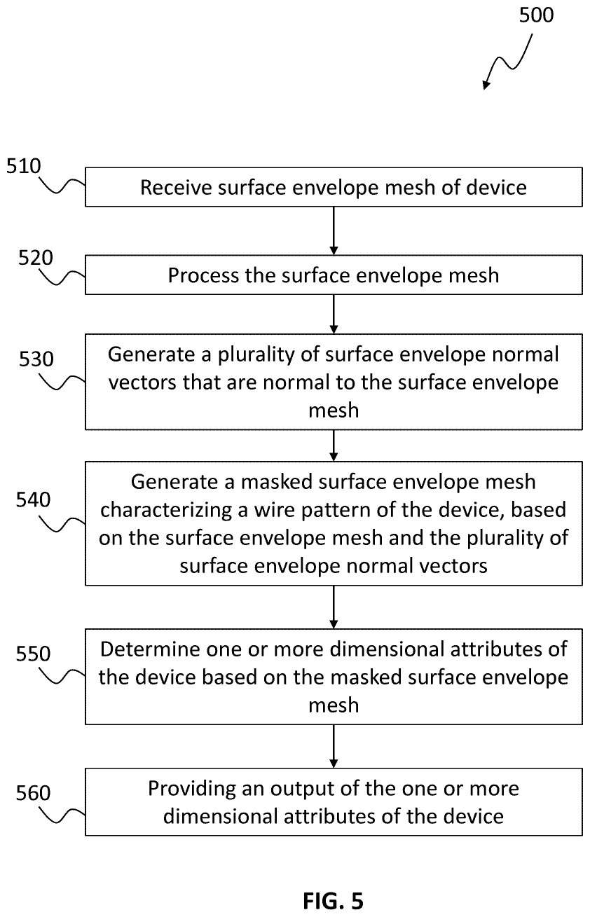

Generally, as shown in FIG. 5, an example method 500 of measuring dimensional attribute(s) of a stent device can include receiving a surface envelope mesh of the stent device 510, processing the surface envelope mesh 520, and generating a plurality of surface envelope normals that are normal to the surface envelope mesh 530. The method 500 can further include generating a masked surface envelope mesh 540, where the mask provides information regarding which cells of the surface envelope correspond to wire of the stent device and which cells correspond to open pores of the stent device. Furthermore, the method can include determining one or more dimensional attributes of the device based on the masked surface envelope mesh 550, and providing an output of the one or more dimensional attributes of the device 560. In some variations, the method 500 can be used to measure one or more dimensional attributes of the stent device including porosity, coverage factor, pore size, pore density, braid angle, pitch, and/or amount of foreshortening of the stent device.

### 1. Receiving a Surface Envelope Mesh of Device

As shown in FIG. 5, the method 500 of measuring dimensional attributes of a stent device includes receiving a surface envelope mesh of the stent device 510. The manner in which the surface envelope mesh can vary depending on the nature of the stent device. For example, in some variations, a stent device of interest can be a physical device, and the method 500 may be performed to obtain real-world measurements of the stent device. As another example, in some variations, method 500 may be performed to obtain theoretical measurements of the device.

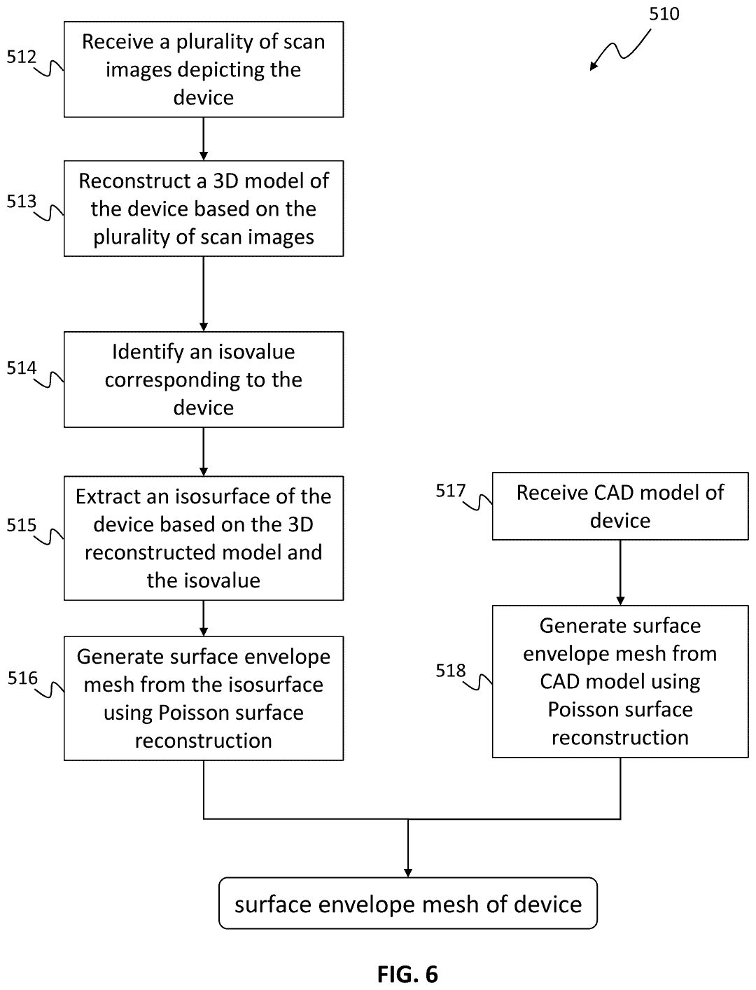

As shown in FIG. 6, in some variations where the stent device is available as a physical device, a surface envelope mesh of the stent device can be obtained at least in part by receiving a plurality of scan images depicting the device 512, and reconstructing a 3D model of the device based on the scan images 513. For example, the physical stent device can be scanned with any suitable imaging medium such as computed tomography (CT) (e.g., microCT), as 2D image stacks that can be rendered as a 3D volume reconstruction of the stent device, or can be scanned with 3D imaging techniques. Given a 3D reconstruction of the stent device, the method 500 can further include identifying an isovalue corresponding to the stent device 514, extracting an isosurface of the stent device 514, and generating the surface envelope mesh from the isosurface 516. More specifically, in some variations, the isosurface can be extracted using a suitable isocontouring algorithm such as the flying edges algorithm (see Schroeder et al., “Flying Edges: A High-Performance Scalable Isocontouring Algorithm”, 2015 IEEE 5^{th} Symposium on Large Data Analysis and Visualization, October 2015). Once the isosurface is extracted, it can be used in block 516 to generate the surface envelope mesh using Poisson surface reconstruction. For example, the surface envelope mesh can be extracted using a Poisson surface reconstruction algorithm (see, e.g., Kazhdan et al., “Poisson Surface Reconstruction”, Eurographics Symposium on Geometry Processing (2006)). In some variations, Poisson disk sampling can be used prior to surface reconstruction to obtain uniformly distributed samples and/or remove noise (see, e.g., Yuksel, C., “Sample Elimination for Generating Poisson Disk Sample Sets”, Computer Graphics Forum, Volume 34, Issue 2 (May 2015)).

Additionally or alternatively, as shown in FIG. 6, in some variations the surface envelope mesh can be received as a direct input (if available) into the dimensional attribute measurement module, or generated from a CAD model of the stent device that is provided as an input into the dimensional attribute measurement module. For example, receiving a surface envelope mesh of the stent device 510 can include receiving a CAD model of the stent device 517, and generating the surface envelope mesh from the CAD model 518. In some variations, the surface envelope mesh can be generated from the CAD model using Poisson surface reconstruction, similar to that described above (with respect to block 516).

### 2. Processing the Surface Envelope Mesh

As shown in FIG. 5, in some variations the method 500 can include processing the surface envelope mesh 520 prior to further analysis. For example, in some variations, processing the surface envelope mesh 520 can include smoothing the surface envelope mesh 520 to reduce noise (e.g., reduce “bumpiness” of the surface). An example of a suitable smoothing algorithm is the Taubin smoothing algorithm (see, Taubin, G., “Curve and Surface Smoothing Without Shrinkage,” IBM Research Report RC-19536 (September 1994)), though other smoothing algorithms may additionally or alternatively be used to smooth the surface envelope mesh (e.g., Gaussian smoothing).

As another example, in some variations, processing the surface envelope mesh 520 can include refining the cell size to sufficiently capture wire detail of the braid wire forming the stent device. For example, the surface envelope mesh can be processed by subdividing the surface envelope mesh until cell sizes are a predetermined size relative to the diameter of the braid wire (e.g., for metrological purposes, at least between about 4 times to about 10 times smaller than the wire diameter, or at least about 4 times smaller than the wire diameter, or at least about 6 times smaller than the wire diameter, or at least about 8 times smaller than the wire diameter, or at least about 10 times smaller than the wire diameter, or at least about 12 times smaller than the wire diameter).

### 3. Generating a Plurality of Surface Envelope Normals

As shown in FIG. 5, the method 500 can further include generating a plurality of surface envelope normal vectors that are normal to the surface envelope mesh 530. In some variations, generating the plurality of surface envelope normal vectors can be similar to that described above with respect to the block 410 of the method 400 for modeling a braided stent device. More specifically, in some variations, generating a plurality of surface envelope normal vectors can include implementing a version of Hoppe's algorithm (see Hoppe et al., “Surface Reconstruction from Unorganized Points”, ACM SIGGRAPH Computer Graphics, Volume 26, Issue 2, 71-78 (Jul. 1, 1992)).

### 4. Generating a Masked Surface Envelope Mesh

As shown in FIG. 5, the method 500 can further include generating a masked surface envelope mesh 540. The masked surface envelope mesh functions to characterize a wire pattern of the device, based on a normal projection of wires onto the surface envelope mesh. Accordingly, in some variations the masked surface envelope mesh can be generated based on the surface envelope mesh and the plurality of surface envelope normal vectors.

For example, in some variations, generating the masked surface envelope mesh 540 can include projecting the surface envelope normal vectors (e.g., outwards from the surface, or inwards from the surface) a predetermined distance via ray tracing. The surface envelope normals can be projected as rays a distance equal to at least between about 1-5 times the diameter of the braid wire (e.g., at least one wire diameter, at least two wire diameters, at least three wire diameters, at least four wire diameters, or at least five wire diameters). If a ray projected from a surface envelope location intersects a wire, then the surface envelope cell at that location is given one value (e.g., 1) to indicate that the cell includes wire, and otherwise if the ray does not intersect a wire then the surface envelope cell at that location is given another value (e.g., 0) to indicate that the cell includes an open pore. By repeating this cell value assignments across the surface envelope mesh, a mask of cell values that represent the existence of wire cells or open cells. In other words, the mask includes a plurality of cells including one or more wire cells (where the projected surface envelope normals intersect with the representation of wires of the stent device) and one or more open cells (where the projected surface envelope normals do not intersect with the representation of wires of the stent device).

### 5. Determining One or More Dimensional Attributes

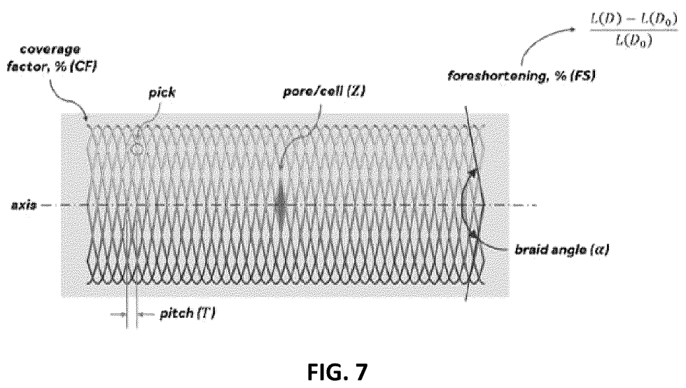

Given the masked surface envelope mesh, which contains information on which cells include wire and which cells do not include wire, the method 500 can further include determining one or more dimensional attributes of the device using the masked surface envelope mesh 550. For example, the one or more dimensional attributes of the stent device can include porosity, coverage factor, pore size, pore density, braid angle, pitch, and/or amount of foreshortening of the stent device. Many of these dimensional attributes are illustrated in FIG. 7, which is a schematic of a braided stent including an array of pores or cells (Z), where each pore or cell has outer boundary edges that intersect or cross at various pick locations.

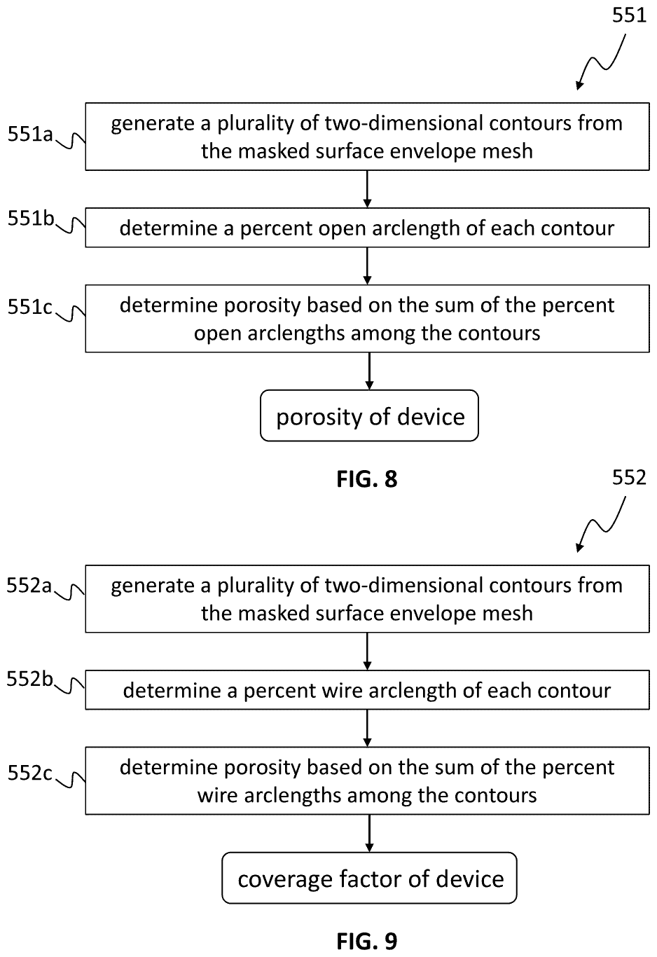

FIG. 8 is a schematic flowchart showing an example variation of determining porosity of the stent device 551. Porosity can be defined as the percent surface area of the stent envelope that is not covered by stent material (wire). As shown in FIG. 8, determining porosity of the stent device can include generating a plurality of 2D contours 551a from the masked surface envelope mesh (e.g., by slicing the envelope normal to the longitudinal device axis to create contours). Each contour can then be isolated using connectivity analysis. Determining porosity 551 can further include, for each contour, determining a percent open arclength of the contour 551b (i.e., portion of the contour that is non-wire) and determining porosity as the sum of the percent open arclengths among the contours 551c.

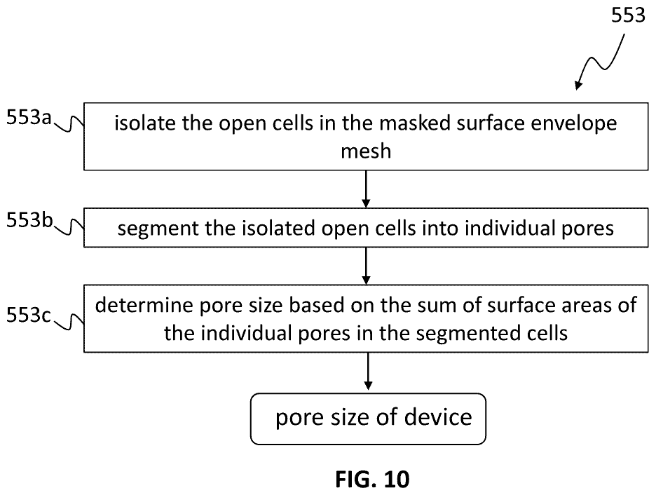

FIG. 9 is a schematic flowchart showing an example variation of determining coverage factor (e.g., Metal Surface Area) of the stent device 552. Coverage factor can be defined as the percent surface area of the stent envelope covered by stent material (wire). Coverage factor is the inverse of porosity. As shown in FIG. 9, determining coverage factor 552 can include generating a plurality of 2D contours 552a from the masked surface envelope mesh (e.g., by slicing the envelope normal to the longitudinal device axis to create contours). Each contour can then be isolated using connectivity analysis. Determining coverage favor 552 can further include, for each contour, determining a percent wire arclength of the contour 552b (i.e., portion of the contour that is wire) and determining coverage factor as the sum of the percent wire arclengths among the contours 552c.

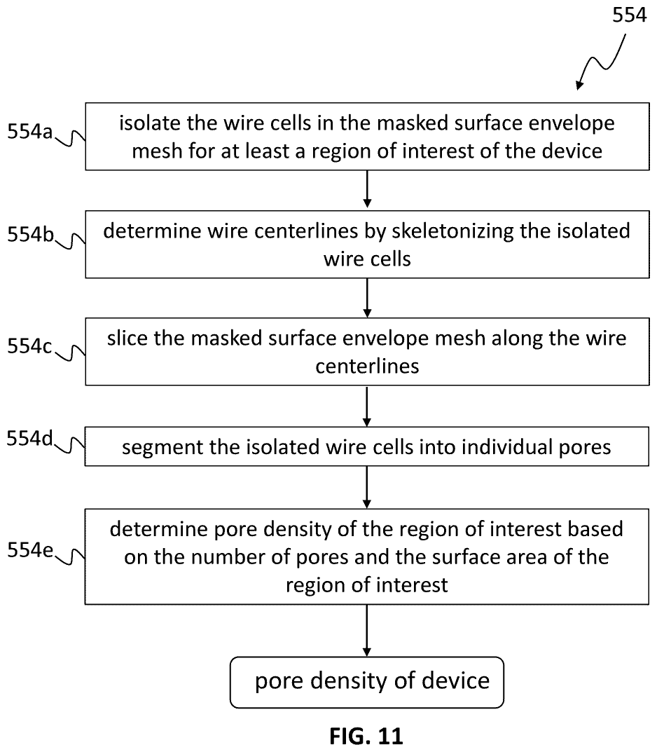

FIG. 10 is a schematic flowchart showing an example variation of determining pore size of the stent device 553. Pore size can be defined as the area of the minimal surface bounded by a stent cell. As shown in FIG. 10, determining pore size 553 can include isolating the open cells in the masked surface envelope mesh 553a (e.g., by removing the wire cells from the mask, leaving only the open/pore cells), segmenting the isolated open cells into individual pores 553b using connectivity analysis, and determining pore size as the sum of surface areas of the individual pores in the segmented cells 553c.

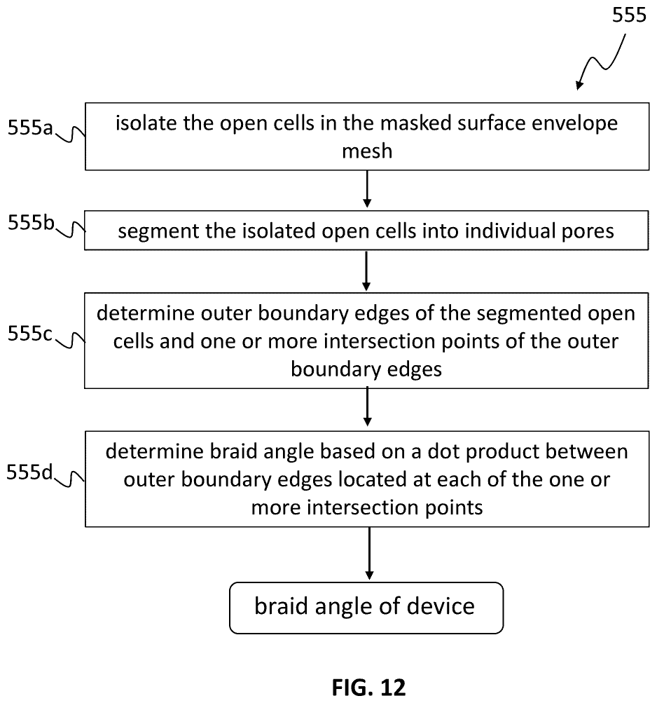

FIG. 11 is a schematic flowchart showing an example variation of determining pore density of at least a region of interest of the stent device 554. Pore density can be defined as the number of stent cells per unit surface area on the stent, and can vary throughout the surface of the stent. Accordingly, pore density of one region of the stent device can be different from pore density in another region of the stent device. As shown in FIG. 11, determining pore density of a region of interest of the stent device 554 can include isolating the wire cells in the masked surface envelope mesh for at least a region of interest of the device 554a (e.g., by removing the open/pore cells from the mask, leaving only the wire cells), determining wire centerlines by skeletonizing the isolated wire cells 554b, slicing the masked surface envelope mesh along the wire centerlines 554c, segmenting the isolated wire cells into individual pores 554d using connectivity analysis, and determining pore density of the region of interest based on the number of pores and surface area of the region of interest 554e (e.g., number of pores divided by the surface area).

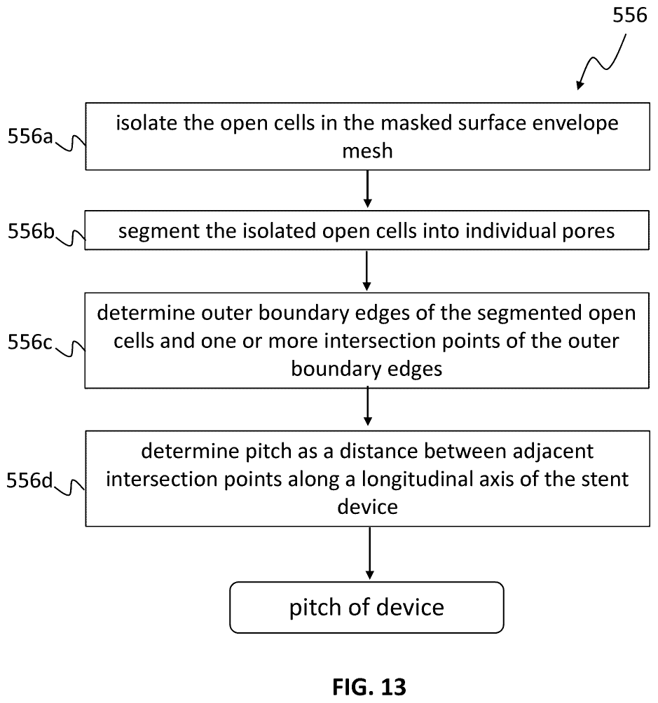

FIG. 12 is a schematic flowchart showing an example variation of determining braid angle of the stent device 555. Braid angle can be defined as the angle between crossing wires relative to a stent axis (e.g., longitudinal or central axis of the stent). As shown in FIG. 12, determining braid angle 555 can include isolating the open cells in the masked surface envelope mesh 555a (e.g., by removing the wire cells from the mask, leaving only the open/pore cells), segmenting the isolated open cells into individual pores 555b using connectivity analysis, determining outer boundary edges of the segmented open cells and one or more intersection points of the outer boundary edges 555c (“corners” of the open cells), and determining braid angle as a dot product between outer boundary edges located at each of the one or more intersection points (sharing a “corner” of an open cell).

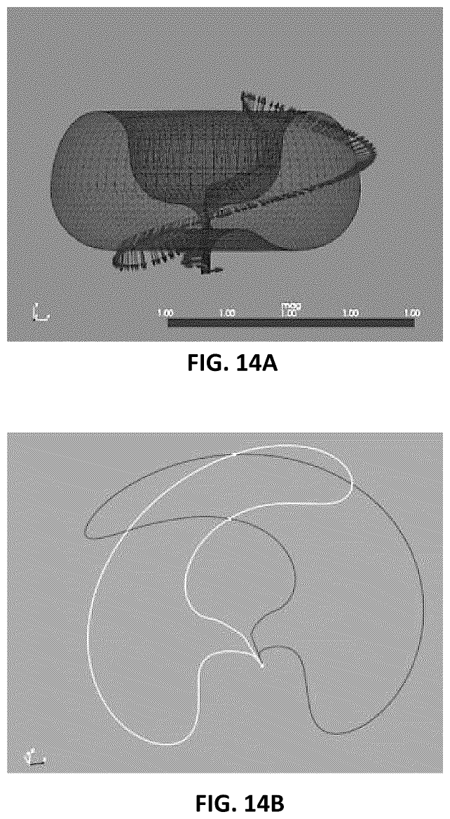

FIG. 13 is a schematic flowchart showing an example variation of determining pitch of the stent device 556. Pitch can be defined as the distance between wire crossings, and is often measured in picks per inch (PPI). As shown in FIG. 13, determining pitch of the stent device 556 can include isolating the open cells in the masked surface envelope mesh 556a (e.g., by removing the wire cells from the mask, leaving only the open/pore cells), segmenting the isolated open cells into individual pores 556b using connectivity analysis, determining outer boundary edges of the segmented open cells and one or more intersection points of the outer boundary edges 556c (“corners” of the open cells), and determining pitch as a distance between adjacent intersection points along a longitudinal axis of the stent device 556d.

In some variations, the method 500 can further include determining foreshortening of the stent device. Foreshortening can be defined as the percent change in stent length per unit change in stent diameter from its original length and diameter.

### 6. Providing an Output of the One or More Dimensional Attributes

As shown in FIG. 5, the method 500 can further include providing an output of the one or more dimensional attributes of the stent device 560. For example, the output can be provided to a display (e.g., for viewing by a user) and/or communicated to a separate software application for further use (e.g., CAD software, FEA, manufacturing device such as 3D printing, etc.).

In some variations, the output can include numerical data including numerical written measurement of dimensional attribute(s) of the stent device. For example, the dimensional attribute measurement data (and any notable conclusions regarding certain regions of the interest of the device, etc.) can be provided in a report in any suitable document format (e.g., Microsoft Word, PDF, LaTEX). In some variations, the method can include automatically formatting the measurement data in accordance with a predetermined format, such as by using a templating syntax algorithm. Additionally or alternatively, an artificial intelligence language model can be used to generate a written text summary of the measurement data for easier readability and consumption by a user (and/or to conform with any report template formatting requirements, etc.). Additionally or alternatively, measurement data can be provided in any suitable graphical format (e.g., histogram that shows distribution of attribute values throughout the stent device), and/or statistical analysis of the measurement data (e.g., mean, median, mode, standard deviation, etc.) can be provided.

Additionally or alternatively, the output can include a visual representation of the stent device. For example, the output can include an image of the stent device (e.g., braid model, CAD model, etc.) that is color-mapped on a pore-by-pore basis (or other spatial resolution) to a scale of measurement values (e.g., scalar bar). The image can be a static image, or can include an animation (e.g., GIF). In some variations, the image can be manipulable by a user through the user interface (e.g., rotate, zoom in, zoom out, click and drag, etc.) to allow inspection of the measurement values distributed around various regions of the stent device.

In some variations, the output can additionally or alternatively have a file format suitable for further analysis by CAD software, FEA software (e.g., ABAQUS input deck), ANSYS or other fluid dynamics package.

However, the output of the dimensional attributes of the stent device can be provided in any suitable manner.

### III. EXAMPLES

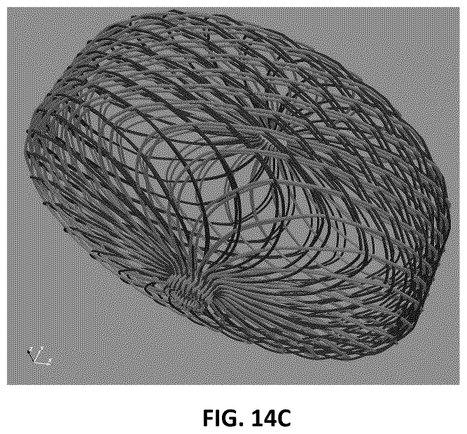

FIGS. 14A through 14C illustrate portions of an example implementation of the method for modeling a braided stent device (e.g., method 400) as described above. The method 400 was performed to model a braided stent device having a non-developable surface. FIG. 14A depicts a surface envelope mesh of the stent device along with a set of surface normals that are normal to the surface envelope (e.g., generated through the processes described herein with respect to block 412). FIG. 14B depicts wire weave paths of the braid (e.g., wire centerline paths) generated through the processes described herein with respect to blocks 420, 422, and 424. FIG. 14C depicts an output of the model of the braided stent device (e.g., generated through the processes described herein with respect to block 470).

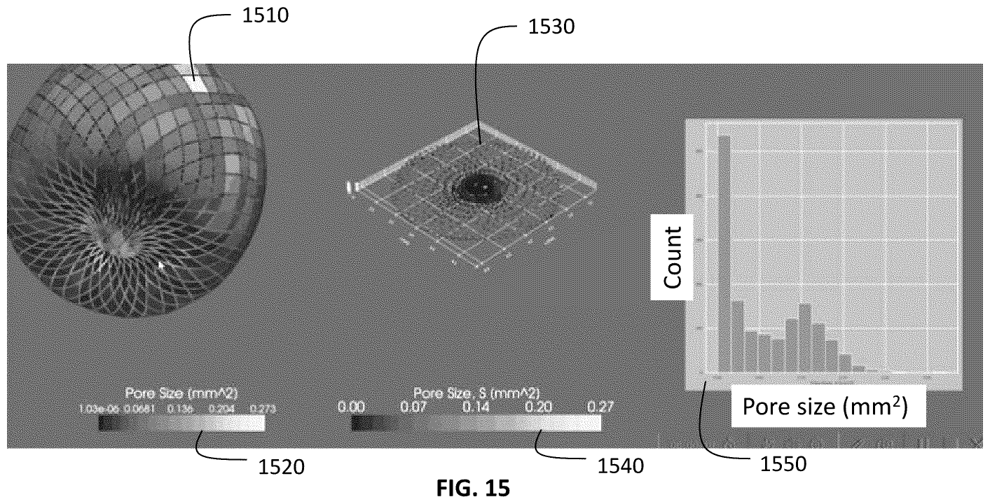

FIG. 15 illustrates various example outputs of measured pore size of a stent device (e.g., measured using the method 500 as described above). The method 500 was performed to characterize a braided stent device having a non-developable surface. FIG. 15 illustrates an example display including a three-dimensional, manipulable (and/or animated) image 1510 of the stent device with pores whose pore sizes are color-mapped to a scalar bar 1520. The example display also includes a two-dimensional cross-sectional view 1530 of the stent device with pores whose pore sizes are similarly color-mapped to a scalar bar 1540. A distribution of pore size in the stent device is displayed in a histogram 1550.

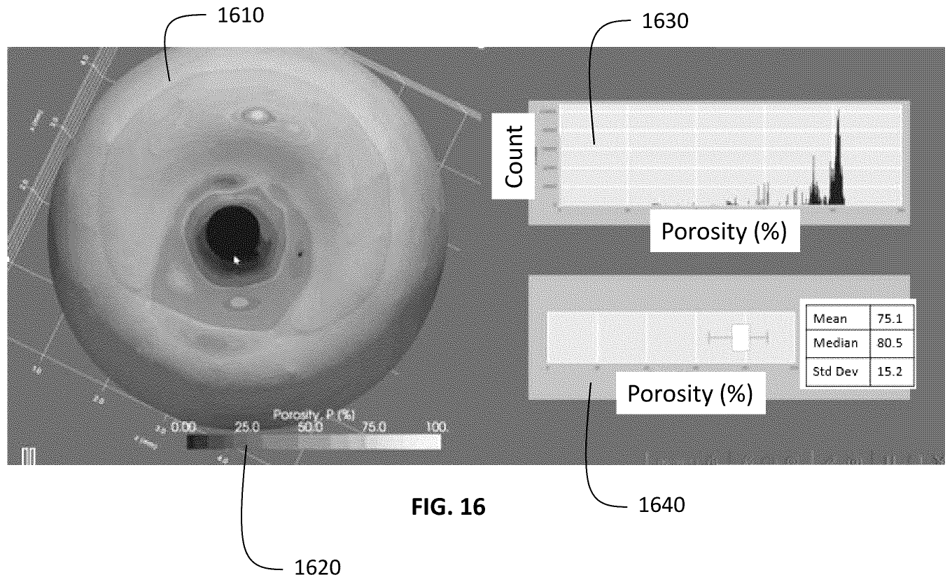

FIG. 16 illustrates various example outputs of measured porosity of a stent device (e.g., measured using the method 500 as described above). The method 500 was performed to characterize a braided stent device having a non-developable surface. FIG. 16 illustrates an example display including a three-dimensional, manipulable (and/or animated) image 1610 of the stent device whose porosity is color-mapped to a scalar bar 1620. The example display also includes a distribution of porosity in the stent device in the form of a histogram 1630, and other statistical information (mean, median, and standard deviation) of the porosity throughout the stent device in the form of a box and whisker plot 1640.

### CONCLUSION

Although many of the embodiments are described above with respect to systems, devices, and methods for the modeling and measurement of stent devices, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other variations without several of the features shown and described above with reference to FIGS. 3-16.

The descriptions of variations of the technology are not intended to be exhaustive or to limit the technology to the precise form disclosed above. Where the context permits, singular or plural terms may also include the plural or singular term, respectively. Although specific variations of, and examples for, the technology are described above for illustrative purposes, various equivalent modifications are possible within the scope of the technology, as those skilled in the relevant art will recognize. For example, while steps are presented in a given order, alternative variations may perform steps in a different order. The various variations described herein may also be combined to provide further variations.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific variations have been described herein for purposes of illustration, but that various modifications may be made without deviating from the technology. Further, while advantages associated with certain variations of the technology have been described in the context of those variations, other variations may also exhibit such advantages, and not all variations need necessarily exhibit such advantages to fall within the scope of the technology. Accordingly, the disclosure and associated technology can encompass other variations not expressly shown or described herein.

I/We claim:

1. A method for modeling a braided stent device, the method comprising:
receiving a surface envelope mesh of the stent device;
receiving at least one helicoid corresponding to a braid pitch of the stent device;
generating a plurality of wire centerline paths based on the surface envelope mesh and the helicoids;
generating a plurality of wire intersection points corresponding to intersections of the wire centerline paths;
generating a plurality of wire intersection normal vectors that are normal to the surface envelope mesh at the wire intersection points;
shaping the wire centerline paths by warping the wire intersection points along the wire intersection normal vectors in accordance with a desired braid pattern; and
generating a braid model of the stent device based on the shaped wire centerline paths and a desired wire diameter.

2. The method of claim 1, wherein the at least one helicoid surface comprises a right-hand helicoid and a left-hand helicoid.

3. The method of claim 2, wherein generating the plurality of wire centerline paths comprises determining intersections the surface envelope mesh and the helicoid surfaces.

4. The method of claim 3, further comprising modifying the wire centerline paths to include ordered, equispaced wire centerline points.

5. The method of claim 4, wherein generating the plurality of wire intersection normal vectors comprises:
generating a plurality of surface envelope normal vectors that are normal to the surface envelope mesh; and
defining the plurality of wire intersection normal vectors as a subset of surface envelope normal vectors located at the wire intersection points.

6. The method of claim 5, wherein generating a plurality of surface envelope normal vectors comprises:
determining, for each point on the surface envelope mesh, a best fit plane through the point that minimizes the distance between the point and the k-nearest neighbors of the point;
determining, for each point on the surface envelope mesh, a surface envelope normal vector that is normal to the best fit plane corresponding to the point; and
orienting the surface envelope normal vectors such that all of the surface envelope normal vectors point inward or outward relative to the surface envelope mesh.

7. The method of claim 6, wherein generating a braid model of the stent device comprises sweeping wire surfaces with the desired wire diameter around the shaped wire centerline paths.

8. A method for characterizing a braided stent device, comprising:
receiving a surface envelope mesh of the stent device, the surface envelope mesh comprising representations of wires of the stent device;
generating a plurality of surface envelope normal vectors that are normal to the surface envelope mesh;
generating a masked surface envelope mesh characterizing a wire pattern of the device, based on the surface envelope mesh and the plurality of surface envelope normal vectors; and
determining one or more dimensional attributes of the stent device based on the masked surface envelope mesh.

9. The method of claim 8, wherein generating a plurality of surface envelope normal vectors comprises:
determining, for each point on the surface envelope mesh, a best fit plane through the point that minimizes the distance between the point and the k-nearest neighbors of the point;
determining, for each point on the surface envelope mesh, a surface envelope normal vector that is normal to the best fit plane corresponding to the point; and
orienting the surface envelope normal vectors such that all of the surface envelope normal vectors point inward or outward relative to the surface envelope mesh.

10. The method of claim 9, wherein generating a masked surface envelope mesh comprises:
projecting the surface envelope normal vectors via ray tracing;
generating a mask of cells comprising a plurality of wire cells and a plurality of open cells,
wherein the plurality of wire cells are located where the projected surface envelope normal vectors intersect with the representation of wires of the stent device, and
wherein the plurality of open cells are located where the projected surface envelope normal vectors do not intersect with the representation of wires of the stent device.

11. The method of claim 10, wherein determining one or more dimensional attributes of the stent device comprises determining at least one of: porosity, coverage factor, pore size, pore density, braid angle, pitch, or amount of foreshortening of the stent device.

12. The method of claim 11, wherein determining one or more dimensional attributes of the stent device comprises determining porosity by:
generating a plurality of two-dimensional contours from the masked surface envelope mesh that are normal to a longitudinal axis of the device;
for each contour, determining a percent open arclength of the contour, the percent open arclength corresponding to one or more open cells of the masked surface envelope mesh in the contour; and
determining porosity of the stent device based on the sum of the percent open arclengths among the contours.

13. The method of claim 12, wherein determining one or more dimensional attributes of the stent device comprises determining coverage factor by:
generating a plurality of two-dimensional contours from the masked surface envelope mesh that are normal to a longitudinal axis of the device;
for each contour, determining a percent wire arclength of the contour, the percent wire arclength corresponding to one or more wire cells of the masked surface envelope mesh in the contour; and
determining porosity of the stent device based on the sum of the percent wire arclengths among the contours.

14. The method of claim 13, wherein determining one or more dimensional attributes of the stent device comprises determining pore size by:
isolating the open cells in the masked surface envelope mesh;
segmenting the isolated open cells into individual pores of the stent device using connectivity analysis; and
determining pore size based on the sum of surface areas of the individual pores in the segmented cells.

15. The method of claim 14, wherein determining one or more dimensional attributes of the stent device comprises determining pore density of a region of interest by:
isolating the wire cells in the masked surface envelope mesh for at least the region of interest;
determining wire centerlines of at least the region of interest by skeletonizing the isolated wire cells;
slicing the masked surface envelope mesh along the wire centerlines;
segmenting the isolated wire cells into individual pores of the stent device using connectivity analysis; and
determining pore density of the region of interest based on the number of pores in the region of interest divided by the surface area in the region of interest.

16. The method of claim 15, wherein determining one or more dimensional attributes of the stent device comprises determining braid angle of the stent device by:
isolating the open cells in the masked surface envelope mesh;
segmenting the isolated open cells into individual pores of the stent device using connectivity analysis; and
determining outer boundary edges of the segmented open cells and one or more intersection points of the outer boundary edges;
determining the braid angle based on a dot product between outer boundary edges located at each of the one or more intersection points.

17. The method of claim 16, wherein determining one or more dimensional attributes of the stent device comprises determining pitch of the stent device by:
isolating the open cells in the masked surface envelope mesh;
segmenting the isolated open cells into individual pores of the stent device using connectivity analysis;
determining outer boundary edges of the segmented open cells and one or more intersection points of the outer boundary edges; and
determining pitch as a distance between adjacent intersection points along a longitudinal axis of the stent device.

18. The method of claim 17, further comprising generating the surface envelope mesh from imaging data and providing an output comprising the one or more dimensional attributes of the stent device.

19. The method of claim 18, wherein the output comprises at least one of a written report, a graphical display of the stent device, a computer-aided drawing model, or a data format for finite element analysis.

20. The method of claim 19, wherein the stent device is an intravascular stent device.
